(19) United States
(12) Patent Application Publication
(10) Pub. No.: US 2017/0304769 A1
(43) Pub. Date: Oct. 26, 2017

(54) METHOD FOR PURIFYING BIOGAS THROUGH MEMBRANES AT NEGATIVE TEMPERATURES

(71) Applicant: L'Air Liquide, Societe Anonyme pour I'Etude et I'Exploitation des Procedes Georges Claude, Paris (FR)

(72) Inventors: Sarah BIGEARD, Sassenage (FR); Delphine GARNAUD, Grenoble (FR); David J. HASSE, Middletown, DE (US); Sudhir S. KULKARNI, Wilmington, DE (US); Edgar S. SANDERS, Newark, DE (US); Golo ZICK, Fontaine (FR)

(73) Assignee: L'Air Liquide, Societe Anonyme pour I'Etude et I'Exploitation des Procedes Georges Claude, Paris (FR)

(21) Appl. No.: 15/507,967

(22) PCT Filed: Aug. 12, 2015
(86) PCT No.: PCT/FR2015/052197

§ 371 Date: Mar. 1, 2017

(30) Foreign Application Priority Data
Sep. 3, 2014 (FR) 1458225

(51) Int. Cl. B01D 53/22; B01D 53/26; F25J 3/08; C10L 3/10; B01D 53/00

(52) U.S. Cl. CPC B01D 53/229; B01D 53/226; B01D 53/227; B01D 53/26; B01D 53/265; C10L 3/104; C10L 3/106; B01D 53/002; F25J 3/08; C10L 2290/06; C10L 2290/08; C10L 2290/548; F25J 2205/80; F25J 2215/60; F25J 2220/66; F25J 2220/68; F25J 2270/08; F25J 2230/30

(57) ABSTRACT

The invention relates to a method for membrane permeation of a gas flow including methane and carbon dioxide, wherein said gas flow is cooled to a temperature of 0° C. to −60° C. before being fed into a membrane separation unit.

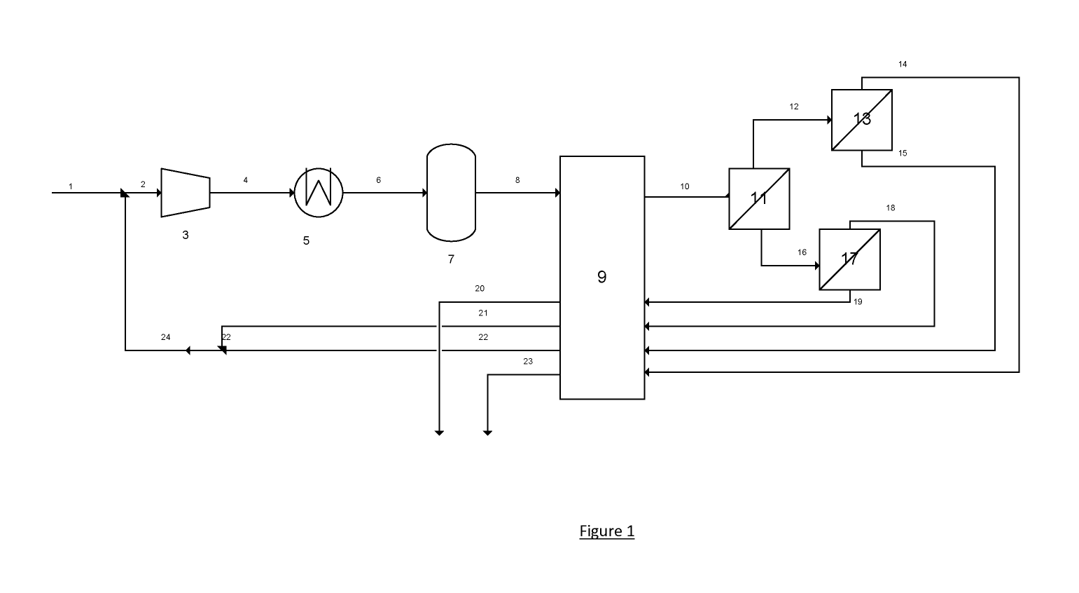

### CROSS REFERENCE TO RELATED APPLICATIONS

This application is a §371 of International PCT Application PCT/FR2015/052197, filed Aug. 12, 2015, which claims the benefit of FR1458225, filed Sep. 3, 2014, both of which are herein incorporated by reference in their entireties.

### FIELD OF THE INVENTION

The present invention relates to a membrane permeation process for a gas stream containing at least methane and carbon dioxide in order to produce a methane-enriched gas stream.

In particular, it relates to biogas purification, with the objective of producing biomethane in accordance with the specifications for injection into a natural gas network.

### BACKGROUND

Biogas is the gas produced during the degradation of organic matter in the absence of oxygen (anaerobic fermentation), also referred to as methanization. This may be a natural degradation—it is thus observed in marshes or municipal waste landfill sites—but the production of biogas may also result from the methanization of waste in a dedicated reactor, referred to as a methanizer or digester.

Due to its main constituents—methane and carbon dioxide—biogas is a potent greenhouse gas; at the same time it is also a significant renewable energy source in the context of the increasing scarcity of fossil fuels.

Biogas predominantly contains methane (CH₄) and carbon dioxide (CO₂) in proportions that vary as a function of the production method, but also, in smaller proportions, water, nitrogen, hydrogen sulphide, oxygen, and also other organic compounds, in trace amounts.

Depending on the organic matter degraded and the techniques used, the proportions of the components differ, but on average biogas comprises, as dry gas, from 30% to 75% methane, from 15% to 60% CO₂, from 0 to 15% nitrogen, from 0 to 5% oxygen and trace compounds.

Biogas is upgraded in various ways. It may, after slight treatment, be upgraded in the vicinity of the production site in order to provide heat, electricity or a mixture of both (cogeneration); the high content of carbon dioxide reduces its heating value, increases the compression and transport costs and limits the economic advantages of upgrading it to this local use.

A more thorough purification of the biogas enables a broader use thereof, in particular a thorough purification of the biogas makes it possible to obtain a biogas that is purified to the specifications of natural gas and which could be substituted therefor. Biogas thus purified is “biomethane”. Biomethane thus supplements natural gas resources with a renewable portion produced at the heart of territories; it can be used for exactly the same uses as natural gas of fossil origin. It may supply a natural gas network or a vehicle filling station and it may also be liquefied in order to be stored in the form of liquefied natural gas (LNG), etc.

The methods of upgrading biomethane are determined as a function of local contexts: local energy requirements, possibilities of upgrading as biomethane fuel, existence nearby of networks for distributing or transporting natural gas in particular. Creating synergies between the various operators working in a territory (farmers, manufacturers, public authorities), the production of biomethane helps territories to acquire greater energy self-sufficiency.

The purification of biogas to give biomethane mainly consists of the separation of the CO₂ and of the CH₄. Polymer membranes therefore represent a perfectly suitable technology for the separation: indeed, the permeance of CO₂ is much greater than that of CH₄. There are therefore many biogas purification processes that use membranes, and these processes have, with respect to the competing technologies (amine washing, water washing, PSA), three main advantages: availability, compactness of the membranes and their flexibility of use. Although this technology makes it possible to achieve high methane recovery rates, while ensuring the quality of the biomethane produced, it nevertheless has two main limits:
the electricity consumption is relatively high (i.e. ≧0.25 kWh/Nm³ crude biogas), due to two parameters: the operating pressure and the degree of recycling of a portion of the permeate necessary for achieving high yields;
the number of membranes may be high (for example for a 4-stage membrane treating 750 Nm³/h of crude biogas, it is possible to use 18 modules (each module contains more than a million fibres)).

Specifically, the intrinsic performances of polymer membranes (permeance, selectivity) are limited, and the selectivity of these materials between CO₂ and CH₄ requires both a relatively high operating pressure, and a multi-stage purification, with a stream recycled upstream of the compressor. Moreover, since the performances of polymer membranes are restricted by the Robeson curve, a high selectivity, chosen to limit methane losses, requires a limited productivity, which increases the number of membranes necessary for treating a given stream of biogas.

Starting from here, one problem that is faced is to provide an improved biogas purification process, this is to say that has a lower electricity consumption and that uses a smaller number of membranes compared to a process from the prior art.

### SUMMARY OF THE INVENTION

One solution of the present invention is a process for purifying a gas stream comprising methane and carbon dioxide by membrane permeation, in which process the gas stream is cooled to a temperature between 0° C. and −60° C. before being introduced into a membrane separation unit.

Depending on the case, the process according to an embodiment of the invention may have one or more of the following features:
the gas stream is cooled to a temperature between −20° C. and −45° C. before being introduced into the membrane separation unit;
said process comprises the following successive steps: a step (a) of compressing the gas stream to a pressure between 5 and 20 bar, a first step (b) of cooling the compressed gas stream to a temperature between 0° C. and 15° C., a step (c) of drying the cooled and compressed gas stream (i.e. that makes it possible to obtain a water content ≦0.1 ppm), a second step (d) of cooling the gas stream resulting from step (c) by means of a heat exchanger to a temperature between 0° C. and −60° C., a step (e) of separating the gas stream resulting from step (d) through at least one membrane stage so as to obtain a CO₂-enriched permeate and a CO₂-depleted retentate, a step (f) of recovering a methane-enriched gas stream;
said process comprises a preliminary membrane separation step between step (c) and step (d), preferably using a CO₂-permeable membrane;
the separation step (e) involves first, second and third membrane stages that each provide a CO₂-depleted retentate and a CO₂-enriched permeate, with the first stage receiving the gas stream resulting from step (d), the second stage receiving the retentate from the first membrane and third membrane receiving the permeate from the first stage;
step (f) of recovering a methane-enriched gas stream comprises a first sub-step of recovering the retentate from the second stage and a second sub-step of reheating the retentate from the second stage to a temperature between 0° C. and 20° C.;
the retentate from the second stage is reheated and then is sent to a liquefaction unit;
the reheating of the retentate from the second stage is carried out by means of the exchanger;
after step (e) the permeate from the second stage and the retentate from the third stage are recovered before reheating them in the exchanger to a temperature between 0° C. and 20° C. and then mixing them with the gas stream to be purified before the compression step (a);
the permeate from the second stage and the retentate from the third stage are reheated in the exchanger to different temperatures;
after step (e) the permeate from the third stage is reheated to a temperature between 0° C. and 20° C. before sending it to a vent or to a vent treatment system;
after step (e) the permeate from the third stage is reheated before sending it to a liquefaction unit.

The crude biogas, purified of its impurities (NH₃, H₂S, VOCs), composed of CH₄ (45%-65%), CO₂ (35%-55%), O₂ (0-5%) and N₂ (0-5%) and dried sufficiently thoroughly (i.e. until a dew point of −5° C. is obtained) in order to prevent the water in the system freezing, is compressed to between 5 and 20 bar. It is then cooled by an air heater and/or exchanger containing iced water to a temperature between 0° C. and 15° C. After final drying, either it enters directly into an exchanger in which it is cooled to a temperature between 0° C. and −60° C., or this exchanger is preceded by a first membrane stage between 0° C. and 15° C. The cooled gas is then sent to one or more membrane stages, in parallel or in series. Each module produces a methane-rich fraction, referred to as retentate, and a CO₂-rich fraction, referred to as permeate. The gas stream most enriched in methane (greater than 90% CH₄) is referred to as biomethane. It is sent to the exchanger, where it is reheated to a temperature between 0° C. and 20° C. The gas stream most depleted in methane (between 0 and 10% CH₄) passes into the exchanger where it is reheated to between 0° C. and 20° C., and is then sent to the vent or to a vent treatment system. The other gas streams produced by the membrane modules are sent to the exchanger or they are reheated to between 0° C. and 20° C., and then recycled to upstream of the compressor. Another advantageous configuration is to take out one or more of the streams leaving the exchanger at a cold enough temperature to achieve a thermal integration, for example for precooling of the crude biogas.

The process makes it possible to achieve a methane yield of between 90% and 99.99%, and to produce a biomethane for which the methane purity is greater than 97%. The discharge pressure of the compressor that makes it possible to achieve thermal self-sufficiency of the process is between 5 and 15 bar.

Another subject of the present invention is a plant for purifying a gas stream comprising methane and carbon dioxide by membrane permeation, said plant comprising an exchanger that makes it possible to cool the gas stream to a temperature between 0° C. and −60° C., and a membrane separation unit downstream of the exchanger.

Preferably, the exchanger makes possible to cool the gas stream to a temperature between −20° C. and −45° C.

The plant according to an embodiment of the invention preferably can include, in the flow direction of the gas stream:
(a) a compressor that is configured to compress the gas stream to between 5 and 20 bar,
(b) a cooling means that is configured to cool the gas stream to a temperature between 0° C. and 15° C.,
(c) a dryer that is configured to dry the cooled and compressed gas stream so as to obtain a gas stream having a water content of less than 0.1 ppm,
(d) an exchanger that is configured to cool the gas stream to a temperature between 0° C. and −60° C.,
(e) a separation unit comprising at least one membrane stage more permeable to carbon dioxide that is configured to separate the gas stream leaving the exchanger.

### BRIEF DESCRIPTION OF THE DRAWINGS

These and other features, aspects, and advantages of the present invention will become better understood with regard to the following description, claims, and accompanying drawing(s). It is to be noted, however, that the drawing(s) illustrate only several embodiments of the invention and are therefore not to be considered limiting of the invention's scope as it can admit to other equally effective embodiments.

The FIGURE shows an embodiment of the invention.

### DETAILED DESCRIPTION OF THE INVENTION

The invention will now be described in greater detail with the aid of the FIGURE which is a diagram of the plant according to invention.

The crude biogas 1, containing 43.6% CO₂, 54.6% CH₄, 0.8% N₂ and 0.2% O₂, saturated with water, at 5° C. and at a pressure of 0.1 barg, is mixed with the recycled stream 24, containing 66.6% CO₂. The stream 2 is then sent to the compressor 3, where it is compressed to 9.6 barg, before being cooled to 5° C. After cooling, the water is removed in a separator, then the gas is reheated up to 15° C. The stream of gas 6 is then sent to the dryer 7. The stream 8 of dry gas, containing 51.2% CO₂, then passes through the exchanger, in which it is cooled to −30° C. The stream of cooled gas enters into a first membrane state, where it is separated into two fractions. The retentate 12 is depleted in CO₂ and contains no more than 30% CO₂, it is sent to a second membrane stage. The permeate 16 is enriched in CO₂ and contains 90% CO₂, it is sent to a third membrane stage. The second membrane stage in turn produces two fractions, the stream 14 depleted to 1.3% CO₂, and the stream 15 enriched to 73% CO₂. The third membrane stage also produces two fractions, the stream 18 depleted to 38% CO₂, and the stream 19 enriched to 99.3% CO₂. The CO₂-rich stream 19 is reheated in the exchanger 9 from −30° C. to 25° C., and then sent to the vent. The stream 14, referred to as biomethane, contains 99.5% of the methane contained in the crude biogas 1, and is reheated to 13.4° C. and then sent to its final use (injection into the network, or fuel gas for vehicles). The streams 15 and 18 are heated to 13.4° C., mixed and sent to upstream of the compressor 3.

Compared to a similar process according to the prior art at ambient temperature, this process makes it possible to reduce the number of membranes and the specific electricity consumption, and if necessary the operating pressure. This is what the table below shows:

| | Operating | | Specific electricity |
|---|---|---|---|
| | pressure | Number | consumption |
| | (bar) | of membranes | (kWh/Nm3) |
| Conventional process | 12 | 18 | 0.24 |
| at ambient T | | | |
| Cold membranes | 10 | 7 | 0.207 |

Depending on the desired applications, the stream of biomethane and/or the vent stream may be produced at a temperature below ambient temperature, in order to be sent to liquefaction units, thus reducing the electricity consumption of the latter.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1-13. (canceled)

14. Process for purifying a gas stream comprising methane and carbon dioxide by membrane permeation, said process comprising the following successive steps:
a) compressing the gas stream to a pressure between 5 and 20 bar;
b) a first step of cooling the compressed gas stream to a temperature between 0° C. and 15° C.;
c) drying the cooled and compressed gas stream to obtain a water content ≦0.1 ppm;
d) a second step of cooling the gas stream resulting from step c) using a heat exchanger to a temperature between 0° C. and −60° C.;
e) separating the gas stream resulting from step d) through at least one membrane stage so as to obtain a CO₂-enriched permeate and a CO₂-depleted retentate; and
f) recovering a methane-enriched gas stream.

15. The process according to claim 14, wherein the gas stream is cooled to a temperature between −20° C. and −45° C. before being introduced into the membrane separation unit.

16. The process according to claim 14, wherein said process comprises a preliminary membrane separation step between step (c) and step (d).

17. The process according to claim 14, wherein the separation step (e) involves first, second and third membrane stages that each provide a CO₂-depleted retentate and a CO₂-enriched permeate, with the first stage receiving the gas stream resulting from step (d), the second stage receiving the retentate from the first stage and third stage receiving the permeate from the first stage.

18. The process according to claim 17, wherein step f) of recovering a methane-enriched gas stream comprises a first sub-step of recovering the retentate from the second stage and a second sub-step of reheating the retentate from the second stage to a temperature between 0° C. and 20° C.

19. The process according to claim 18, wherein the reheating of the retentate from the second stage is carried out by means of the exchanger.

20. The process according to claim 17, wherein the retentate from the second stage is reheated and then is sent to a liquefaction unit.

21. The process according to claim 20, wherein the reheating of the retentate from the second stage is carried out by means of the exchanger.

22. The process according to claim 17, wherein after step e) the permeate from the second stage and the retentate from the third stage are recovered before reheating them in the exchanger to a temperature between 0° C. and 20° C. and then mixing them with the gas stream to be purified before the compression step a).

23. The process according to claim 22, the permeate from the second stage and the retentate from the third stage are reheated in the exchanger to different temperatures.

24. The process according to claim 17, wherein after step e) the permeate from the third stage is reheated to a temperature between 0° C. and 20° C. before sending it to a vent or to a vent treatment system.

25. The process according to claim 17, wherein after step e) the permeate from the third stage is reheated before sending it to a liquefaction unit.

26. A plant for purifying a gas stream comprising methane and carbon dioxide by membrane permeation, said plant comprising, in the flow direction of the gas stream:
a) a compressor configured to compress the gas stream to between 5 and 20 bar,
b) a cooling means configured to cool the gas stream to a temperature between 0° C. and 15° C.,
c) a dryer configured to dry the cooled and compressed gas stream so as to obtain a gas stream having a water content of less than 0.1 ppm,
d) an exchanger configured to cool the gas stream to a temperature between 0° C. and −60° C.,
e) a separation unit comprising at least one membrane stage more permeable to carbon dioxide configured to separate the gas stream leaving the exchanger.

27. The purification plant according to claim 26, wherein the exchanger is configured to cool the gas stream to a temperature between −20° C. and −45° C.
